(19) United States
(12) Patent Application Publication
(10) Pub. No.: US 2021/0060137 A1
(43) Pub. Date: Mar. 4, 2021

(54) NUTRIENT COMPOSITIONS HAVING ENHANCED BIOAVAILABILITY

(71) Applicant: Rainbow Light Nutritional Systems, LLC, Oakland, CA (US)

(72) Inventors: Steven Craig Bobzin, Santa Cruz, CA (US); Michele L. McRae, Santa Cruz, CA (US); Lisa C. Offringa, Santa Cruz, CA (US); Susan L. Trimbo, Boca Raton, FL (US)

(21) Appl. No.: 17/011,760

(22) Filed: Sep. 3, 2020

Related U.S. Application Data
(60) Provisional application No. 62/895,560, filed on Sep. 4, 2019.

(51) Int. Cl. A61K 38/43; A61K 9/16; A61K 33/30; A61K 33/26; A61K 31/355; A61K 31/593; A61K 31/714; A61K 31/525; A61K 31/197; A61K 47/46; A61K 35/747; A61K 35/745; A61K 35/742; A23L 33/135; A23L 33/105; A23L 33/155; A23L 33/165

(52) U.S. Cl. CPC A61K 38/43; A23V 2002/00; A61K 33/30; A61K 33/26; A61K 31/355; A61K 31/593; A61K 31/714; A61K 31/525; A61K 31/197; A61K 47/46; A61K 35/747; A61K 35/745; A61K 35/742; A23L 33/135; A23L 33/105; A23L 33/155; A23L 33/165; A61K 9/16

(57) ABSTRACT

Disclosed are nutrient compositions formulated to provide enhanced bioavailability when administered by or taken by a subject. A nutrient composition includes one or more nutrients such as vitamins, minerals, micronutrients, amino acids, peptides, proteins, enzymes, plant extracts, botanicals, and/or herbal ingredients. At least a portion of the nutrients is comprised of one or more micronized nutrients having an average particle size of about 1 μm to about 100 μm. At least a portion of the mineral component is comprised of one or more chelated minerals. The composition also includes an absorption-promoting probiotic.

### CROSS-REFERENCE TO RELATED APPLICATION

The application claims the benefit of U.S. Provisional Patent Application Ser. No. 62/895,560 filed on Sep. 4, 2019, entitled NUTRIENT COMPOSITIONS HAVING ENHANCED BIOAVAILABILITY, which is incorporated herein in its entirety by reference.

### BACKGROUND

Multivitamin supplements are generally formulated to provide daily amounts of vitamins, minerals, and other nutrients to the user. Such supplementation may be carried out to ensure adequate levels of essential nutrients in the diet. This may be particularly important for those on restricted diets, for those with a condition that depletes nutrients, for those with poor diets, or for pregnant or breast-feeding women, for example. Even for those who do not have one of these conditions, multivitamins remain popular as a simple and straightforward way to ensure that they are obtaining adequate levels of important nutrients.

However, even though a multivitamin may contain given amounts of nutrients, it does not necessarily follow that the nutrients, when orally consumed, will be adequately absorbed by the body to be available for biological use where needed. If the bioavailability of the multivitamin is low, many of the nutrients may pass through the gastrointestinal tract unabsorbed to be excreted and wasted, even if there was a biological need for the nutrients. A supplement may compensate for poor absorption by increasing the amounts of the poorly absorbed nutrients in an effort to compensate for the poor bioavailability of the nutrients. However, this approach is wasteful and does nothing to improve the underlying causes of the waste.

There is therefore a continuing need for nutrient compositions, including multivitamin compositions such as daily multivitamin compositions, that are formulated to provide more effective absorption and bioavailability.

### BRIEF SUMMARY

The present disclosure is directed to nutrient compositions, including multivitamins and “once-a-day” multivitamins, formulated to provide enhanced bioavailability. That is, the nutrient compositions described herein are formulated to beneficially provide one or more of: less waste of nutrients in a supplement formulation, use of smaller doses to provide the same level of absorption as compared to conventional supplement formulations, greater absorption of one or more nutrients for a given dose as compared to conventional supplement formulations, and/or more effective treatment or supplementation to a subject in need of the nutrient composition.

In one embodiment, a nutrient composition comprises a nutrient component and an absorption-promoting probiotic. The nutrient component comprises one or more micronized nutrients that have been micronized to an average particle size of about 1 μm to about 100 μm, preferably about 2 μm to about 80 μm, or about 3 μm to about 60 μm, or about 4 μm to about 40 μm, or about 5 μm to about 20 μm. The one or more micronized nutrients may include a variety of vitamins and/or minerals. In a particular embodiment, the micronized nutrients comprise one or more of vitamin D₃, vitamin E, vitamin B₂, vitamin B₅, vitamin B₁₂, zinc, or iron.

The absorption-promoting probiotic may comprise one or more bacteria from the genera Bacillus, Lactobacillus, Bifidobacterium, or other probiotic known to improve intestinal health and/or the integrity of the intestinal endothelial layer. A preferred embodiment includes the bacterium Bacillus coagulans. The absorption-promoting probiotic is provided in an amount of at least 30 million CFU, such as at least about 50 million CFU, at least about 75 million CFU, at least about 100 million CFU, at least about 250 million CFU, at least about 500 million CFU, at least about 1 billion CFU, or at least about 2 billion CFU. By way of example, the nutrient composition may be provided in a dosage form where each dosage form unit (e.g., each tablet, each capsule, etc.) is formulated to provide the absorption-promoting probiotic in an amount within the foregoing ranges. An upper limit may be about 5 or 10 billion CFU, though such an upper limit may be a factor of product economics more than product efficacy.

A nutrient composition may also include a plant extract, a botanical component, an herbal component, a digestive support component, and/or one or more excipients. The nutrient composition may be provided in a dosage form that provides about 0.05 to 12 times the RDA of the one or more nutrients of the nutrient component per dosage form unit.

A nutrient composition as described herein may also include one or more of amino acids, peptides, proteins, enzymes, micronutrients, phytonutrients, and fatty acids. Plant extracts may be prepared from whole plants, or from one or more sub portions of the plants such as fruits, roots, flowers, leaves, and stems. Phytonutrients may include carotenoids, xanthophylls, fatty acids, alkaloids, terpenoids, and phenylpropanoids.

One or more minerals included in the nutrient composition may be provided in chelated form. For example, the one or more chelated minerals comprise one or more of calcium, iron, magnesium, zinc, selenium, chromium, potassium, copper, manganese, and molybdenum.

The nutrient composition may be provided in a dosage form such as tablets, capsules, powders, gummies, food products, food additives, beverages, beverage additives, candies including chocolates and suckers, pastilles, dietetically acceptable sprays, or gels. Multiple dosage form units may be provided in a bulk container such as a carton, box, can, jar, bag, pouch, bottle, or jug.

It is to be understood that both the foregoing general description and the following detailed description are exemplary and explanatory only and are intended to provide further explanation of the invention as claimed.

Further features and advantages of the present invention will become apparent to those of ordinary skill in the art in view of the detailed description of preferred embodiments below.

### BRIEF DESCRIPTION OF THE DRAWINGS

To further clarify the above and other advantages and features of the present invention, a more particular description of the invention will be rendered by reference to specific embodiments thereof which are illustrated in the drawings located in the specification. It is appreciated that these drawings depict only typical embodiments of the invention and are therefore not to be considered limiting of its scope. The invention will be described and explained with additional specificity and detail through the use of the accompanying drawings in which:

FIG. 1 is a graph comparing area under the curve (AUC) values of an enhanced bioavailability supplement to a control supplement, where the control supplement is similar to the enhanced bioavailability supplement but omits an absorption-promoting probiotic and does not include any micronized nutrients, showing that the enhanced bioavailability supplement provided enhanced bioavailability for the vast majority of measured nutrients; and

FIG. 2 is a graph comparing maximum concentration (Cₘₐₓ) values of the enhanced bioavailability supplement to the control supplement, showing that the enhanced bioavailability supplement provided higher Cₘₐₓ values for the vast majority of measured nutrients.

### DETAILED DESCRIPTION

### Introduction

Described herein are nutrient compositions formulated to provide enhanced bioavailability when taken by or administered to a user. The effective bioavailability of the compositions described herein may beneficially enable one or more of: less waste of nutrients in a supplement formulation, the use of smaller doses to provide the same level of absorption as compared to conventional supplement formulations, greater absorption of one or more nutrients for a given dose as compared to conventional supplement formulations, and/or more effective treatment or supplementation to a subject in need of the nutrient composition.

### Definitions

Before describing the present invention in detail, it is to be understood that this invention is not limited to particularly exemplified systems or process parameters that may, of course, vary. It is also to be understood that the terminology used herein is for the purpose of describing particular embodiments of the invention only, and is not intended to limit the scope of the invention in any manner.

All publications, patents and patent applications cited herein, whether supra or infra, are hereby incorporated by reference in their entirety to the same extent as if each individual publication, patent or patent application was specifically and individually indicated to be incorporated by reference.

The term “comprising” which is synonymous with “including,” “containing,” or “characterized by,” is inclusive or open-ended and does not exclude additional, unrecited elements or method steps.

The term “consisting essentially of” limits the scope of a claim to the specified materials or steps and those that do not materially affect the basic and novel characteristic(s) of the claimed invention.

The term “consisting of” as used herein, excludes any element, step, or ingredient not specified in the claim.

It must be noted that, as used in this specification and the appended claims, the singular forms “a,” “an” and “the” include plural referents unless the content clearly dictates otherwise. Thus, for example, reference to a “surfactant” includes one, two or more surfactants.

Numbers, percentages, ratios, or other values stated herein may include that value, and also other values that are about or approximately the stated value, as would be appreciated by one of ordinary skill in the art. A stated value should therefore be interpreted broadly enough to encompass values that are at least close enough to the stated value to perform a desired function or achieve a desired result, and/or values that round to the stated value. The stated values include at least the variation to be expected in a typical manufacturing or formulation process, and may include values that are within 10%, within 5%, within 1%, etc. of a stated value. Furthermore, the terms “substantially,” “similarly,” “about,” or “approximately,” as used herein represent an amount or state close to the stated amount or state that still performs a desired function or achieves a desired result. For example, the term “substantially” “about” or “approximately” may refer to an amount that is within 10% of, within 5% of, or within 1% of, a stated amount or value.

Some ranges may be disclosed herein. Additional ranges may be defined between any values disclosed herein as being exemplary of a particular parameter. All such ranges are contemplated and within the scope of the present disclosure.

The phrase “free of” or similar phrases if used herein means that the composition or article comprises 0% of the stated component, that is, the component has not been intentionally added. However, it will be appreciated that such components may incidentally form thereafter, under some circumstances, or such component may be incidentally present, e.g., as an incidental contaminant.

The phrase “substantially free of” or similar phrases as used herein means that the composition or article preferably comprises 0% of the stated component, although it will be appreciated that very small concentrations may possibly be present, e.g., through incidental formation, contamination, or even by intentional addition. Such components may be present, if at all, in amounts of less than 1%, less than 0.5%, less than 0.25%, less than 0.1%, less than 0.05%, less than 0.01%, less than 0.005%, less than 0.001%, or less than 0.0001%. In some embodiments, the compositions or articles described herein may be free or substantially free from any specific components not mentioned within this specification.

In the application, effective amounts are generally those amounts listed as the ranges or levels of ingredients in the descriptions, which follow hereto. Unless otherwise stated, amounts listed in percentage (“%'s”) are in weight percent (based on 100% active) of any composition.

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

As used herein, “user,” “subject,” or “patient” refers to any animal requiring therapy, treatment, or prophylaxis, or any animal suspected of requiring therapy, treatment, or prophylaxis. Prophylaxis means that a regiment is undertaken to prevent a possible occurrence, such as where a supplement is taken to avoid a vitamin and/or mineral deficiency even if one is not presently identifiable. “User,” “patient,” and “subject” are used interchangeably herein. Although typical implementations will relate to human administration, a “user,” “subject,” or “patient” may also refer to other members of the animal kingdom, including mammals such as, but not limited to, other primates, rodents, canines, and felines, and also including non-mammals such as fish, reptiles, and birds.

The term “unit dose” refers to a dosage form that is configured to deliver a specified quantity or dose of composition or component thereof. Example dosage forms include, but are not limited to, tablets, capsules, powders, food products, food additives, beverages, beverage additives, candies (e.g., gummies, suckers), pastilles, dietetically acceptable sprays (such as flavored mouth spray), or gels (e.g., gel packs). Such dosage forms may be configured to provide a full unit dose or fraction thereof (e.g., ½, ⅓, or ¼ of a unit dose).

Certain embodiments, for example, provide the nutrient composition in the dosage form of tablets or capsules, and several tablets or capsules may be provided in a bulk container, such as a carton, box, can, jar, bag, pouch, bottle, or jug. Each tablet or capsule within the container may be configured to provide a full unit dose or fraction thereof (e.g., ½, ⅓, or ¼ of a unit dose) when taken.

Another dosage form that can be used to provide a unit dose of composition or component thereof is a unit dose measuring device, such as a cup, scoop, syringe, or spoon, which is configured to hold therein a measured quantity of composition equaling a full unit dose or fraction thereof (e.g., ½, ⅓, or ¼ of a unit dose). For example, a bulk container, such as a carton, box, can, jar, bag, pouch, bottle, or jug, containing several unit doses of composition (e.g., 5-250 or 10-150 unit doses) can be provided to a user together with a unit dose measuring device that is configured to provide a unit dose, or fraction thereof, of composition or component thereof.

A kit for use in providing a composition as disclosed herein in bulk form, while providing unit doses of the composition, may comprise a bulk container holding therein a quantity of composition and a unit dose measuring device configured to provide a unit dose, or fraction thereof, of composition or component thereof. One or more unit dose measuring devices may be positioned inside the bulk container at the time of sale, attached to the outside of the bulk container, prepackaged with the bulk container within a larger package, or provided by the seller or manufacture for use with one or multiple bulk containers.

The kit may include instructions regarding the size of the unit dose, or fraction thereof, and the manner and frequency of administration. The instructions may be provided on the bulk container, prepackaged with the bulk container, placed on packaging material sold with the bulk container, or otherwise provided by the seller or manufacturer (e.g., on websites, mailers, flyers, product literature, etcetera). The instructions for use may include a reference on how to use the unit dose measuring device to properly deliver a unit dose or fraction thereof. The instructions may additionally or alternatively include a reference to common unit dose measuring devices, such as spoons, spatulas, cups, and the like, not provided with the bulk container (e.g., in case the provided unit dose measuring device is lost or misplaced). In such case, a kit may be constructed by the end user when following instructions provided on or with the bulk container, or otherwise provided by the seller regarding the product and how to properly deliver a unit dose of composition, or fraction thereof.

The term “administration” or “administering” is used herein to describe the process in which the disclosed compositions are delivered to a subject. In a typical implementation, a user self-administers the composition by taking it orally. Terms such as “take” or “taking” may be used interchangeably with terms such as “administration” or “administering” (e.g., the user “takes” the nutrient composition via self-administration).

In some embodiments, multiple unit doses of the composition are administered over a period of time. The frequency of administration of the composition can vary depending on any of a variety of factors, such as time since a previous administration, objectives of the supplementation, and the like. The duration of administration of the composition (e.g., the period of time over which the composition is administered), can vary depending on any of a variety of factors, including subject response, desired effect of supplementation, etcetera.

The amount of the composition to be administered can vary according to factors such as the age, sex, and weight of the individual, idiosyncratic responses of the individual, and the like. In typical implementations, a unit dose of the composition includes each component nutrient (e.g., each vitamin and/or mineral) at levels related to a recommended dietary allowance (RDA) for each component nutrient (where RDA is used herein as synonymous with reference daily intake (RDI) and with daily value (DV)). For example, each component nutrient may be provided in an amount that is about 0.05 to 12 times the RDA, or about 0.1 to 8 times the RDA, for the component nutrient. RDA values are provided by the United States National Institutes of Health (NIH) Office of Dietary Supplements, and are listed in their Dietary Reference Intake (DRI) reports, among other places. Because RDA values may be updated over time, the RDA values referred to herein are the values listed as of March 2019, which values can be readily determined by one of ordinary skill in the art by reference to the foregoing well-known sources.

A unit dose may be administered once, or may be divided and administered over intervals of time. For example, an embodiment may be formulated as a daily multivitamin intended to be administered substantially every day. Such a daily formulation may be provided in a dosage form that provides the full daily unit dose in a single dosage form (e.g., a single tablet, capsule, etc.), or in a dosage form that divides the full daily unit dose into multiple sub-doses. Where provided in a dosage form that divides the full daily unit dose into multiple sub-doses, the sub-doses may be taken multiple times per day (e.g., two, three, four, or five times per day), such as intervals throughout the day, to arrive at the full daily unit dose.

It is to be understood that administration may be adjusted according to individual need and professional judgment of a person administrating or supervising the administration of the compositions.

As used herein “average particle size” refers to the average diameter or average length along a longest dimension of a sample of particles. The average particle size may be measured as a number length mean, surface area moment mean (i.e., Sauter Mean diameter), or volume moment mean (i.e., De Brouckere mean diameter), for example.

### Nutrient Compositions

In one embodiment, a nutrient composition includes a nutrient component and an absorption-promoting probiotic. The nutrient component may comprise one or more micronized nutrients having an average particle size of about 1 μm to about 100 μm. In contrast to an otherwise similar nutrient component that is not micronized and/or that has an average particle size that is larger than about 100 μm, the micronized nutrients with average particle size of about 1 μm to about 100 μm may provide: 1) greater dissolution of bulk material in the digestive system of the subject (e.g., particularly the stomach); 2) greater absorption in the intestines; or both.

The one or more micronized nutrients more preferably have an average particle size of about 2 μm to about 80 μm, or about 3 μm to about 60 μm, or about 4 μm to about 40 μm, or about 5 μm to about 20 μm.

The absorption-promoting probiotic may beneficially promote digestive health and provide for more effective absorption of nutrients, including vitamins and minerals of the nutrient component. The absorption-promoting probiotic may include one or more microbes associated with digestive health, digestive assistance, the healing of the gut lining, or other health benefits. In one embodiment, the absorption-promoting probiotic comprises one or more bacteria from the genera Bacillus, Lactobacillus, and/or Bifidobacterium. In a preferred embodiment, the absorption-promoting probiotic comprises Bacillus coagulans.

The nutrient composition may be provided in a dosage form (e.g., tablet, capsule, or other dosage form described herein) that includes the absorption-promoting probiotic in an amount of at least about 30 million CFU, such as at least about 50 million CFU or at least about 75 million CFU per each dosage form unit (i.e., per each tablet, capsule, etcetera). More preferably, the absorption-promoting probiotic is included in an amount of at least 1 billion CFU per dosage form unit.

The one or more micronized nutrients may include any mineral and/or vitamin capable of being micronized. For example, the one or more micronized nutrients may be micronized via a mechanical process such as milling (e.g., jet milling) and/or grinding, or via a supercritical fluid micronization process such as the rapid expansion of supercritical solutions (RESS) process, the supercritical anti-solvent (SAS) process, and/or the particles from gas saturated solutions (PGSS) process.

In one embodiment, the one or more micronized nutrients comprise one or more of vitamin D₃, vitamin E, vitamin B₂ (riboflavin), vitamin B₅ (pantothenic acid), vitamin B₁₂, zinc, and iron. Other vitamins or minerals described herein or known in the art may also be provided in a micronized form.

Typically, those vitamins and/or minerals with lower relative bioavailability benefit more from micronization than those that are readily absorbed by the typical subject. For example, some embodiments may include one or more of vitamins A, C, K, B₁ (thiamin), B₃ (niacin), B₆, B₇ (biotin), B₉ (folate), choline, calcium, iodine, magnesium, selenium, copper, manganese, and/or molybdenum in a non-micronized form (e.g., with an average particle size above 50 um or more typically above 100 μm). Although any or all of the foregoing may be micronized, manufacturing costs can be reduced by selectively grouping those nutrients where micronization provides a greater relative effect.

In some embodiments, the nutrient composition may further comprise a digestive support component. The digestive support component may comprise one or more enzymes that aid in the breakdown, conversion, or hydrolysis of ingested food and/or supplements, such as amylase, protease, xylanase, maltase, glucoamylase, hemicellulose, beta-glucanase, phytase, cellulase, alpha-galactosidase, lipase, lactase, and invertase.

The digestive support component may be included in an amount of about 1 mg to about 50 mg, or about 3 mg to about 30 mg, or about 5 mg to about 20 mg. For example, the nutrient composition may be provided in a dosage form that provides the digestive support component in an amount within the foregoing ranges in each dosage form unit.

In some embodiments, the nutrient support component may further comprise a plant extract component. The plant extract component may include, for example, one or more extracts of beet, broccoli, kale, spinach, blackberry, blueberry, carrot, cranberry, pomegranate, citrus fruit, grape, and lutein. The plant extract component may additionally or alternatively include microalgae such as Chlorella and/or Spirulina.

The plant extract component may be included in an amount of about 10 mg to about 500 mg, or about 25 mg to about 350 mg, or about 50 mg to about 250 mg, or about 75 mg to about 200 mg. For example, the nutrient composition may be provided in a dosage form that provides the plant extract component in an amount within the foregoing ranges in each dosage form unit.

In some embodiments, one or more of the minerals included in the nutrient composition are provided in a chelated form. The minerals may be chelated with, for example, short-chain peptides, amino acids (e.g., glycine, arginine, lysine), ethylenediamine, ethylenediaminetetraacetic acid (EDTA), other chelating agents known in the art, or combinations thereof.

Any mineral capable of being chelated and which provides better bioavailability in chelated form may be chelated. In one embodiment, the composition includes one or more of chelated calcium, iron, iodine, magnesium, zinc, selenium, copper, manganese, and molybdenum.

In some embodiments, the composition provides about 0.05 to 12 times the RDA of the one or more nutrients of the nutrient component. For example, the dosage form may be formulated so as to provide 0.05 to 12 times the RDA of the one or more nutrients per each dosage form unit.

### Additional Dosage Form Details

A nutrient composition as described herein may be provided in a dosage form such as tablets, capsules, powders, food products, food additives, beverages, beverage additives, candies including gummies and suckers, pastilles, dietetically acceptable sprays, and gels, for example. Multiple dosage form units may be included in a bulk container such as a carton, box, can, jar, bag, pouch, bottle, or jug.

Certain embodiments may also include one or more excipients. For example, excipients may include coatings, solvents, emulsions, suspensions, syrups, elixirs, dispersion and suspension media, thickening agents, preservatives, fillers, bulking agents, viscosity modifiers, lubricants, stabilizers, sweeteners or flavoring agents, binders, diluents, and/or disintegrants.

Particular examples of excipient ingredients include microcrystalline cellulose, hydroxypropyl cellulose, hydroxypropyl methylcellulose, sodium carboxymethylcellulose, magnesium stearate, stearic acid, silica, dicalcium phosphate, starch, mannitol, lactose, sucrose, cyclodextrins, polyethylene glycol, polyacrylic copolymer.

### Methods of Administration

The nutrient compositions described herein may be administered via any suitable administration route known in the art, including orally, buccally, parenterally, as an injection (subcutaneous, intravenous, intramuscular, intracisternal), nasally, topically, by inhalation, or rectally. Typically, however, the nutrient compositions described herein are formulated for oral administration.

Preferably, a nutrient composition is formulated as a “once-a-day” multivitamin in a dosage form suitable for oral administration and intended for one or two doses per day (though smaller dosages and/or a greater number of doses per day may also be used in certain implementations).

### EXAMPLES

### Example 1

A nutrient composition was prepared according to the amounts illustrated below in Table 1:

TABLE 1

| | Amount | | |
|---|---|---|---|
| | Per Serving | | % DV |
| Vitamin A (as beta-carotene) | 900 | mcg | 100 |
| Vitamin C (as ascorbic acid) | 90 | mg | 100 |
| Vitamin D (as cholecalciferol) | 50 | mcg | 250 |
| Vitamin E (as d-alpha tocopheryl | 15 | mg | 100 |
| succinate) | | | |
| Vitamin K (as phytonadione) | 120 | mcg | 100 |
| Thiamin (as thiamin mononitrate) | 9 | mg | 750 |
| Riboflavin (vitamin B2) | 10 | mg | 769 |
| Niacin (as niacinamide) | 20 | mg | 125 |
| Vitamin B6 (as pyridoxine hydrochloride) | 12 | mg | 706 |
| Folate (as folic acid) | 400 | mcg DFE | 100 |
| | 240 | mcg folic acid | |
| Vitamin B12 (as methylcobalamin) | 10 | mcg | 417 |
| Biotin | 30 | mcg | 100 |
| Pantothenic Acid (as calcium | 5 | mg | 100 |
| pantothenate) | | | |
| Choline (as choline bitartrate) | 55 | mg | 10 |
| Calcium (as citrate) | 100 | mg | 8 |
| Iron (as iron amino acid chelate) | 6 | mg | 33 |
| Iodine (from kelp) | 150 | mcg | 100 |
| Magnesium (as marine magnesium | 100 | mg | 24 |
| extract from sea water) | | | |
| Zinc (as zinc citrate) | 11 | mg | 100 |
| Selenium (as selenomethionine) | 55 | mcg | 100 |
| Copper (as copper amino acid chelate) | 0.9 | mg | 100 |
| Manganese (as manganese citrate) | 2.3 | mg | 100 |
| Molybdenum (as molybdenum amino | 45 | mcg | 100 |
| acid chelate) | | | |
| Vitality Blend | 70 | mg | |
| Organic Pomegranate (fruit) juice powder, | | | |
| Citrus Bioflavonoids, Organic Chlorella, | | | |
| Grape [whole fruit] extract, Lutein | | | |
| Vibrant Blend | 100 | mg | |
| Organic spirulina, organic beet (root), | | | |
| organic broccoli (plant), organic kale | | | |
| (leaf), organic spinach (leaf), organic | | | |
| blackberry (fruit), organic blueberry | | | |
| (fruit), organic carrot (root), organic | | | |
| cranberry (fruit) | | | |
| Digestive Support Blend | 13 | mg | |
| Amylase (90 DU), protease (360 HUT), | | | |
| xylanase (48 XU), maltase (2 DP), | | | |
| glucoamylase (0.2 AGU), hemicellulase | | | |
| (37 HCU), beta-glucanase (0.1 BGU), | | | |
| phytase (0.02 FTU), cellulase | | | |
| (14 CU), alpha-galactosidase | | | |
| (2 GalU), lipase (13 FTP), lactase | | | |
| (4 ALU), invertase (2 SU), Bacillus | | | |
| coagulans (1 Billion CFU) | | | |
| Other ingredients: | | | |
| Microcrystalline cellulose, coating | | | |
| (hydroxypropyl cellulose, hypromellose), | | | |
| hydroxypropyl methylcellulose, | | | |
| magnesium stearate, silica, | | | |
| stearic acid). | | | |

The nutrient composition may be administered to a user as a once-a-day multivitamin supplement. In the nutrient composition, vitamin D₃, vitamin E, vitamin B₂, vitamin B₅, vitamin B₁₂, zinc, and iron were micronized so as to have an average particle size of about 10 μm.

### Example 2

The bioavailability of the supplement of Example 1 was compared to the bioavailability of a control supplement. The control supplement was identical to the supplement of Example 1 other than that it lacked Bacillus coagulans and did not micronize any of the nutrients.

A clinical trial enrolled 12 subjects. For 9 consecutive days, the subjects took the control supplement. On day 10, blood draws were taken from each subject at 0, 1, 2, 4, and 8 hours following the 10^{th} dose, and nutrient levels were measured using the blood draws. The subjects then underwent a 4 day washout period with no dosing. After the washout period, the subjects took the supplement of Example 1 for 9 consecutive days. As with the control supplement, blood draws from each subject were taken on day 10 at 0, 1, 2, 4, and 8 hours following the 10^{th} dose, and nutrient levels were measured using the blood draws.

The blood draw data was used to create a pharmacokinetic curve for each subject when given the control, and for each subject when given the supplement of Example 1. Nutrient levels at the 0 hour point, taken immediately prior to supplementation, were taken as baseline levels and were subtracted from the subsequent measurements. The area under the curve (AUC) of each curve at 8 hours (AUC8) was determined. The Cₘₐₓ of each curve was also determined.

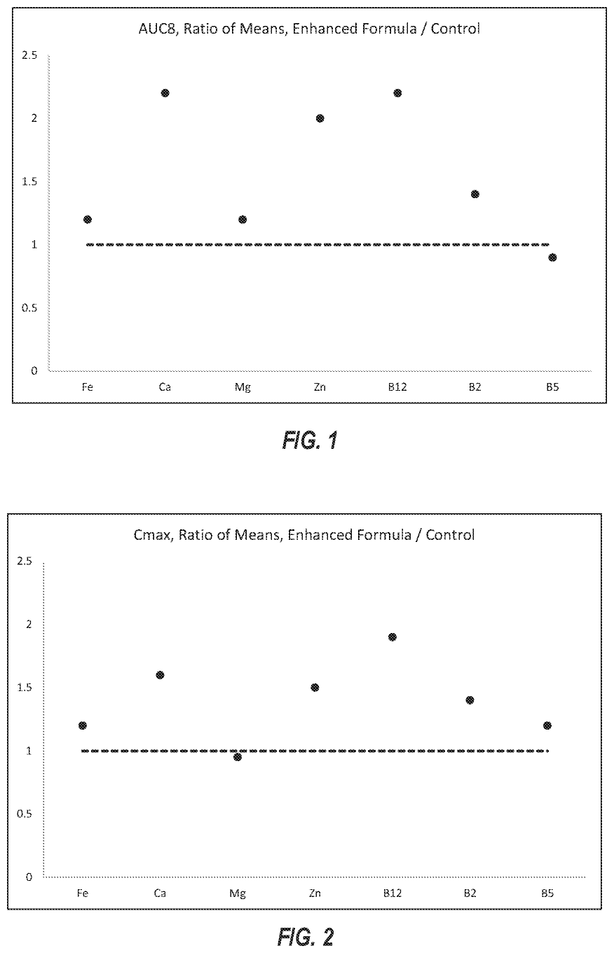

For each nutrient measured, the ratio of the mean AUC8 value for the supplement of Example 1 to the mean AUC8 value for the control supplement was determined. The results are graphically presented in FIG. 1. A value greater than 1 for a given nutrient indicates that the supplement of Example 1 provided a higher mean AUC8 value for that nutrient. As shown, the supplement of Example 1 provided superior mean AUC8 values for the vast majority of nutrients tested.

Similarly, for each nutrient measured, the ratio of the mean Cₘₐₓ value of the supplement of Example 1 to the mean Cₘₐₓ value of the control supplement was determined. The results are graphically presented in FIG. 2. A value greater than 1 for a given nutrient indicates that the supplement of Example 1 provided a higher mean Cₘₐₓ value for that nutrient. As shown, the supplement of Example 1 provided superior Cₘₐₓ values for the vast majority of nutrients tested.

For each tested nutrient, the supplement of Example 1 showed improvement in at least one of AUC8 or Cₘₐₓ as compared to the control supplement.

### Example 3

Vitamin E blood levels were tested during the trial described in Example 2 under the same protocols described in Example 2. The AUC8 values for vitamin E resulting from the supplement of Example 1 were significantly greater (95% confidence interval, grouped using Fisher LSD method) than those of the control supplement.

### Example 4

Trial subjects given the supplement of Example 1 completed the 5-item World Health Organization Well-Being Index (WHO-5) survey. The WHO-5 survey is a widely utilized questionnaire for assessing subjective psychological well-being. The WHO-5 survey scores of the subjects improved to a statistically significant degree (Dunnett's method at 95% confidence interval).

### Example 5

Trial subjects given the supplement of Example 1 completed the Gastrointestinal Symptom Rating Scale (GSRS) survey. The GSRS survey is a widely used survey that measures 15 items combined into symptom clusters depicting reflux, abdominal pain, indigestion, diarrhea, and constipation. Lower scores are better. The GSRS survey scores of the subjects improved to a statistically significant degree (Dunnett's method at 95% confidence interval).

1. A nutrient composition formulated to provide effective bioavailability, the nutrient composition comprising:
a nutrient component comprised of one or more nutrients,
wherein the nutrient component includes one or more micronized nutrients, and
wherein the one or more micronized nutrients have an average particle size of about 1 μm to about 100 μm; and
an absorption-promoting probiotic.

2. The composition of claim 1, wherein the one or more micronized nutrients have an average particle size of about 2 μm to about 80 μm.

3. The composition of claim 1, wherein the one or more micronized nutrients have an average particle size of about 3 μm to about 60 μm.

4. The composition of claim 1, wherein the one or more micronized nutrients have an average particle size of about 4 μm to about 40 μm.

5. The composition of claim 1, wherein the one or more micronized nutrients have an average particle size of about 5 μm to about 20 μm.

6. The composition of claim 1, wherein the absorption-promoting probiotic comprises one or more of Bacillus bacteria, Lactobacillus bacteria, or Bifidobacterium bacteria.

7. The composition of claim 6, wherein the absorption-promoting probiotic comprises Bacillus coagulans.

8. The composition of claim 1, wherein the absorption-promoting probiotic is included in an amount of at least 30 million CFU.

9. The composition of claim 1, wherein the absorption-promoting probiotic is included in an amount of at least 100 million CFU.

10. The composition of claim 1, wherein the absorption-promoting probiotic is included in an amount of at least 250 million CFU.

11. The composition of claim 1, wherein the absorption-promoting probiotic is included in an amount of at least 1 billion CFU.

12. The composition of claim 1, wherein the one or more micronized nutrients comprises one or more of vitamin D, vitamin E, vitamin B₂, vitamin B₅, vitamin B₁₂, zinc, and iron.

13. The composition of claim 1, wherein the nutrient component comprises one or more vitamins or minerals in addition to the one or more micronized nutrients.

14. The composition of claim 1, further comprising a digestive support component comprised of one or more digestive enzymes.

15. The composition of claim 14, wherein the digestive support component comprises one or more of amylase, protease, xylanase, maltase, glucoamylase, hemicellulose, beta-glucanase, phytase, cellulase, alpha-galactosidase, lipase, lactase, and invertase.

16. The composition of claim 14, wherein the composition is provided in a dosage form that provides about 1 mg to about 50 mg, or about 3 mg to about 30 mg, or about 5 mg to about 20 mg of the digestive support component per dosage form unit.

17. The composition of claim 1, further comprising a plant extract component comprised of one or more plant extracts.

18. The composition of claim 17, wherein the plant extract component comprises one or more extracts of beet, broccoli, kale, spinach, blackberry, blueberry, carrot, cranberry, pomegranate, citrus fruit, grape, lutein, Chlorella, and Spirulina.

19. The composition of claim 17, wherein the composition is provided in a dosage form that provides about 10 mg to about 500 mg, or about 25 mg to about 350 mg, or about 50 mg to about 250 mg, or about 75 mg to about 200 mg, of the plant extract component per dosage form unit.

20. A method of administering a nutrient composition, comprising:
providing a nutrient composition as in any one of claims 1 through 26; and
administering the nutrient composition to a user.
